(19) United States
(12) Patent Application Publication
(10) Pub. No.: US 2008/0300687 A1
(43) Pub. Date: Dec. 4, 2008

(54) Medical implantation device for spine

(76) Inventors: Kwan-Ku Lin, Taipei (TW); Larry T. Khoo, Studio City, CA (US)

Correspondence Address: BACON & THOMAS, PLLC, 625 SLATERS LANE, FOURTH FLOOR, ALEXANDRIA, VA, 22314-1176, US

(21) Appl. No.: 11/896,176

(22) Filed: Aug. 30, 2007

(30) Foreign Application Priority Data
May 30, 2007 (TW) 96208894

(51) Int. Cl. A61F 2/44; A61B 17/58

(52) U.S. Cl. 623/17.12; 606/93; 606/94; 623/17.11

(57) ABSTRACT

A medical implantation device for spine is disclosed which includes a flexible container with one end thereof being provided with an infusion end and having one or more layers of flexible peripheral wall; and a tubular component receiving the flexible container with the infusion end and another end of the flexible contained exposing from two ends of the tubular component, and the tubular component is to be implanted between two adjacent spinous processes; wherein the tubular component has an outer diameter greater than a distance between the two adjacent spinous processes, and a medical filling is infused into the flexible container via the infusion end thereof, so that an inflation of a middle section of the flexible container is restricted by the tubular component and the infusion end and the another end of the flexible container are inflated to be movably pressed on the two sides of the two adjacent spinous processes.

### FIELD OF THE INVENTION

The present invention provides a medical implantation device for spine, particularly a medical implant device for spine, which includes a flexible container used singly or jointly with a tubular element for propping two adjacent spinous processes open.

### BACKGROUND OF THE INVENTION

There are a few options in treating a spine disease of which pain occurs at a location where spine nerves are pinched: implanting a pedicle nail, expanding the space between two adjacent vertebrae, expanding and releasing the space where the pinched nerves located; or removing the intervertebral disc between two adjacent vertebrae and replacing the intervertebral disc with an artificial one in order to restore the intervertebral space and the spinal canal space. Alternatively, an “X-stop” or a U-shaped “Co-Flax” spinous process propping-open device is implanted between two adjacent spinous processes from the back of the spine in order to expand the spinal canal space surrounded by the two vertebrae for releasing the pinched nerves and eliminating the pain caused by the pinched nerves.

Currently, the issues taken into consideration during operations include: reducing the area of operation, reducing patient's bleeding, and reducing the chance of infection. Therefore, if the above-mentioned treatments are used merely for releasing the pain caused by the pinched spine nerves, the risk caused by the operation is disproportionate to the patient's problem if no other major spinal problems involved. In particular, a spinal operation often involves major risks. Therefore, the medical field has been constantly investigating how to solve the above-mentioned problems effectively and easily.

In the meantime, US patent publication numbers 2004/0122455, 2004/0210297, and their related patent families, (which are also invented by the inventor of the present invention) disclose spine treatment devices with containers having meshed walls for treating diseases associated with a collapsed vertebra or for propping-up and supporting intervertebral disc space.

In order to achieve the objectives of reducing the area of operation, patient's bleeding and risk of contamination, which are the needs for solving the above-mentioned problems, the present invention provides a medical implant device for spine, by which the operation can be carried out on one side of the spine as much as possible, while taking into account the contour of the spine, effective expansion and formation of stable spinal canal space after operation, etc. As a result, an implant according to the present invention provides the advantages of simple operation procedures, reduced operation time, and faster recovery after operation.

### SUMMARY OF THE INVENTION

One objective of the present invention is to provide a medical implantation device for spine.

Another objective of the present invention is to provide a medical implantation device for spine, which includes a flexible container and/or a tubular element, wherein the flexible container can be inserted into the tubular element first and then the whole set or only the flexible container is implanted between two adjacent spinous processes, thereby providing a propping-open effect and a stable support effect.

Still another objective of the present invention is to provide a medical implantation device for spine, which includes a flexible container and/or a tubular element, wherein the diameter of the cross-section of the tubular element or the diameter of the mid-section of the flexible container after being inflated is larger than the distance between two adjacent spinous processes, wherein the size of the pores on the peripheral wall of the container is smaller than the particle size of the medical filling, and wherein the infusion of the medical filling enables the two ends of the container pressing on the two sides of the spinous processes due to the mid-section of the container being restricted by the tubular element or the mid-section of the container having a different elastic coefficient.

Still a further objective of the present invention is to provide a medical implantation device for spine, which includes a flexible container and/or a tubular element, wherein each section of the flexible container can be pre-formed, and the size of the pores on the peripheral wall is smaller than 100 mesh. Moreover, the invented medical implantation device further includes an infusion tool movably connected to the container. Basically, the medical filling exhibits a slurry form and then becomes solid, such as a bone cement, e.g. poly(methyl methacrylate) (PMMA), or bone substitute, e.g. gypsum, calcium phosphates; or the medical filling exhibits a slurry form and then becomes an elastic semi-solid form, such as silicone filling, for medical use.

The present invention discloses a medical implantation device for spine comprising:

a flexible container with one end thereof being provided with an infusion end, wherein the container has one or more layers of flexible peripheral wall, and the peripheral wall has a plurality of through holes thereon for communicating an inside of the container with an outside of the container; and

a tubular component, wherein the flexible container is inserted into the tubular component with the infusion end and another end of the flexible contained exposing from two ends of the tubular component, and the tubular component is to be implanted between two adjacent spinous processes;

wherein the tubular component has an outer diameter greater than a distance between the two adjacent spinous processes, the through holes on the peripheral wall of the flexible container have a size smaller than a particle size of a medical filling to be infused into the flexible container via the infusion end thereof, wherein an inflation of a middle section of the flexible container is restricted by the tubular component and the infusion end and the another end of the flexible container are inflated to be movably pressed on the two sides of the two adjacent spinous processes, when the medical filling is infused into the flexible container.

The present invention also discloses a medical implantation device for spine comprising a pre-formed stretchable flexible container having an infusion end and another end, wherein the infusion end and the another end of the container are expanded and shrunk to have an expansion coefficient different from that of a middle section of the container, wherein the container comprises one or plural layers of flexible peripheral wall, wherein the peripheral wall is provided with a plurality of through holes for communicating an inside of the container with an outside of the container, and the container is to be implanted between two adjacent spinous processes;

wherein the through holes of the peripheral wall of the flexible container is smaller than a particle size of a medical filling to be infused from the infusion end to inflate the flexible container, wherein a longest distance between two points on a cross-section of the resulting inflated middle section is greater than a distance the two adjacent spinous processes, and the resulting inflated infusion end and the resulting inflated another end of the flexible container are movably pressed on the two sides of the two adjacent spinous processes.

The present invention also discloses a medical implantation device for spine comprising:

a flexible container with one end thereof being provided with an infusion end, wherein the container has one or more layers of flexible peripheral wall, and the peripheral wall has a plurality of through holes thereon for communicating an inside of the container with an outside of the container;

a tubular component, wherein the flexible container is inserted into the tubular component with the infusion end and another end of the flexible contained exposing from two ends of the tubular component, and the tubular component is to be implanted between two adjacent spinous processes; and

a medical filling to be infused into the flexible container via the infusion end thereof,

wherein the tubular component has an outer diameter greater than a distance between the two adjacent spinous processes, the through holes on the peripheral wall of the flexible container have a size smaller than a particle size of the medical filling, wherein the infusion end of the flexible container is sealed following completion of the infusion of the medical filling, the tubular component with the sealed flexible container are implanted between two adjacent spinous processes, and a force is applied on the infusion end to push and move the medical filling from the infusion end to the another end of the flexible container while an inflation of a middle section of the flexible container is restricted by the tubular component, so that the infusion end and the another end of the flexible container are inflated to be movably pressed on the two sides of the two adjacent spinous processes.

The present invention further discloses a medical implantation device for spine comprising:

a pre-formed stretchable flexible container having an infusion end and another end, wherein the infusion end and the another end of the container are expanded and shrunk to have an expansion coefficient different from that of a middle section of the container, wherein the container comprises one or plural layers of flexible peripheral wall, wherein the peripheral wall is provided with a plurality of through holes for communicating an inside of the container with an outside of the container, and the container is to be implanted between two adjacent spinous processes; and

a medical filling to be infused into the flexible container via the infusion end thereof,

wherein the through holes on the peripheral wall of the flexible container have a size smaller than a particle size of the medical filling, wherein the infusion end of the flexible container is sealed following completion of the infusion of the medical filling, the sealed flexible container is implanted between two adjacent spinous processes, and a force is applied on the infusion end to push and move the medical filling from the infusion end to the another end of the flexible container, so that an inflation of a middle section of the flexible container has a longest distance between two points on a cross-section of the inflated middle section greater than a distance between the two adjacent spinous processes, and the infusion end and the another end of the flexible container are inflated to be movably pressed on the two sides of the two adjacent spinous processes.

Preferably, said flexible container is a pre-formed stretchable flexible container, the infusion end and the another end of the container are expanded and shrunk to have an expansion coefficient different from that of a middle section of the container.

Preferably, the size of the through holes on the peripheral wall of the flexible container is smaller than 100 mesh.

Preferably, the medical implantation device for spine of the present invention further comprises an infusion tool disconnectably connected to the infusion end of the flexible container, and the infusion tool is capable of storing and infusing the medical filling.

Preferably, the infusion end of the flexible container and a front end of the infusion tool are provided with threads for disconnectable connection with each other.

Preferably, the medical filling is prepared into a slurry form before operation, infused into the flexible container through an infusion tool, and becomes solid after operation. More preferably, the medical filling is a bone cement for bone tissues or a bone substitute.

Preferably, the medical filling is prepared into a slurry form before operation, infused into the flexible container through an infusion tool, and becomes an elastic semi-solid after operation. More preferably, the medical filling is a silicone filling applicable on bone tissues.

The components in the medical implantation device for spine of the present invention can be the same as the components disclosed in the original invention and related inventions of the present invention, referring to US patent publication numbers 2004/0122455, and 2004/0210297. The components can also be the same as the components disclosed in similar devices, e.g. the prior art mentioned in the above US patent publications: US patent publication numbers 2004/0073308, and 2006/0149379, and U.S. Pat. No. 6,719,773. Furthermore, the definition and use of the peripheral wall and pores disclosed in the above-mentioned flexible container can also be seen in the above-mentioned US patent publication numbers 2004/0122455, and 2004/0210297.

The medical filling in the medical implantation device for spine of the present invention can be a medical material defined in the original and related inventions of the present invention, referring to US patent publication numbers 2004/0122455, and 2004/0210297. It can also be a medical filling disclosed in the similar technologies, e.g. the above-mentioned prior art inventions, US patent publication numbers 2004/0073308, and 2006/0149379, and U.S. Pat. No. 6,719,773. That is the medical filling of the present invention can be prepared into a slurry form prior to the operation and developed into a solid form during/after the operation.

### BRIEF DESCRIPTION OF THE DRAWINGS

FIG. 1 is a schematic diagram showing a medical implantation device for spine constructed according to a first preferred embodiment of the invention.

FIG. 2a and FIG. 2b are schematic diagrams showing the medical implantation device shown in FIG. 1 before and after filling the flexible container 20 with the medical filling 60.

FIG. 3a to FIG. 3c are schematic diagrams of a pre-formed stretchable flexible container of the present invention, which is under pre-inflation shaping, shrinking, and attached with a tubular component, respectively.

FIG. 4a and FIG. 4b are schematic diagrams showing the pre-formed stretchable flexible container shown in FIGS. 3a and 3b being implanted between two adjacent spinous processes according to a first operation method of the present invention.

FIG. 5a and FIG. 5b are schematic diagrams showing the flexible container shown in FIG. 1 being implanted between two adjacent spinous processes according to a first operation method of the present invention.

FIG. 6a and FIG. 6b are schematic diagrams showing the pre-formed stretchable flexible container shown in FIGS. 3a and 3b being implanted between two adjacent spinous processes according to a second operation method of the present invention.

FIG. 7a to FIG. 7e are schematic diagrams showing the operation steps of implanting the medical implantation device for spine of the present invention between two adjacent spinous processes, by which the two adjacent spinous processes are propped open.

### LEGENDS

| | | | |
|---|---|---|---|
| 10 | Medical implantation device for spine | | |
| 20 | Flexible container | 21 | Infusion end |
| 22 | Flexible peripheral wall | 221 | Through hole |
| 23 | Another end | 24 | Middle section |
| 30 | Tubular component | | |
| A | Longest distance on the external | | |
| | wall of tubular component | | |
| 40 | spinous process | | |
| B | distance between two adjacent | | |
| | spinous processes | | |
| 50 | Infusion tool | 51 | Threads |
| 60 | Medical filling | | |
| 70 | Flexible container | 71 | Infusion end |
| 72 | Another end | 73 | Middle section |
| 74 | Flexible peripheral wall | 741 | Through hole |

### DETAILED DESCRIPTION OF THE INVENTION

The present invention is further elaborated by the following preferred embodiments in conjunction with accompanied drawings:

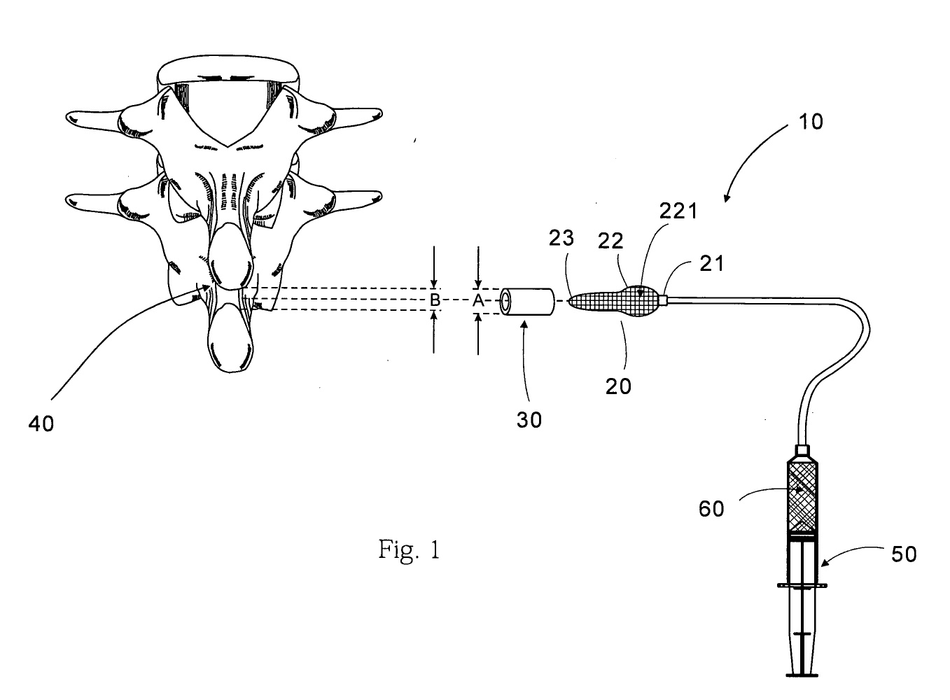

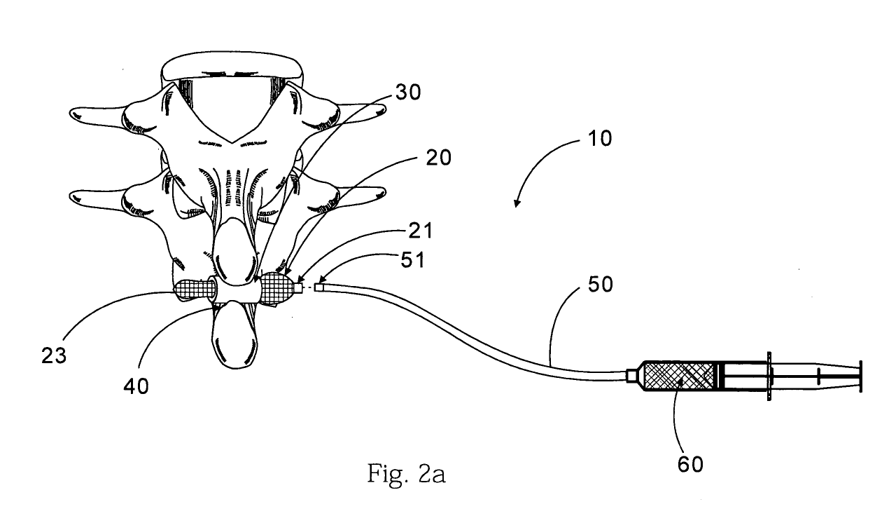

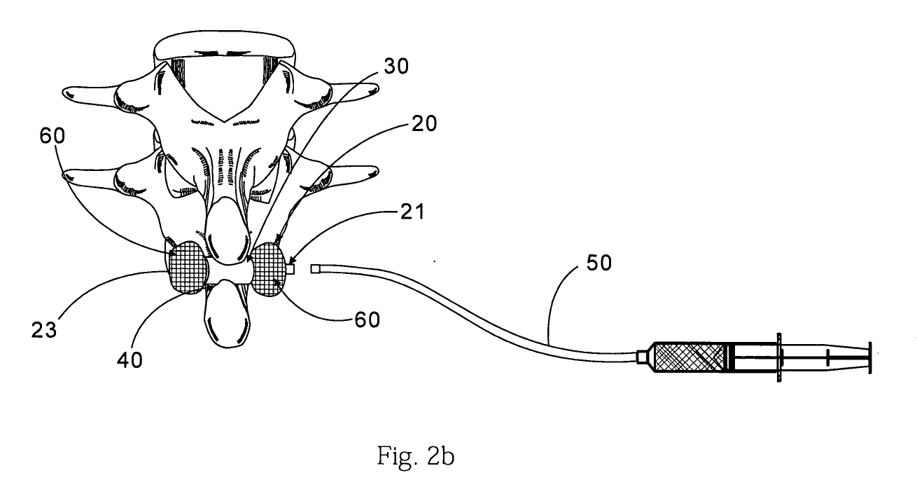

In FIG. 1, FIGS. 2a and 2b, a medical implantation device 10 for spine constructed according to the present invention includes a flexible container 20 and a tubular component 30.

One end of the flexible container 20 is an infusion end 21, and the flexible container 20 includes one or plural layers of flexible peripheral wall 22, wherein the peripheral wall 22 is formed with a plurality of pores 221 thereon for communicating the inside and the outside of the container 20. Furthermore, the flexible container 20 is inserted into the tubular component 30 with the infusion end 21 and the other end thereof exposing from the ends of the tubular component 30. After the container 20 being inserted, the tubular component 30 is implanted between two adjacent spinous processes 40.

The medical implantation device for spine 10 further includes an infusion tool 50, which can be assembled to or disassembled from the infusion end 21 of the flexible container 20 by rotating clockwise or counter-clockwise threads 51 provided thereon. Furthermore, the infusion tool 50 is used to contain and infuse a medical filling 60.

The longest distance A between two points on the external wall of the cross-section of the tubular component 30 is larger than the pitch B of the two adjacent spinous processes implanted with the tubular component 30. Consequently, the medical implantation device 10 for spine is able to perform a propping-up operation after being implanted into the two adjacent spinous processes 40.

The size of the pores 221 on the peripheral wall of the flexible container 20 is smaller than the particle size of the medical filling 60 and is smaller than 100 mesh. The medical filling is a bone cement used for bone tissues, for examples poly(methyl methacrylate) (PMMA), or a bone substitute, such as gypsum, calcium phosphates, etc., or a silicone filling for bone tissues, and is prepared into a slurry form prior to the operation and infused into the flexible container 20 through the infusion tool 50, and will be hardened into solid or elastic semi-solid after the operation.

The medical filling 60 is infused from the infusion end 21 with the middle section of the flexible container 20 being restricted by the tubular component 30, so that the two ends 21, 23 are expanded and movably pressed on the two sides of the spinous process 40.

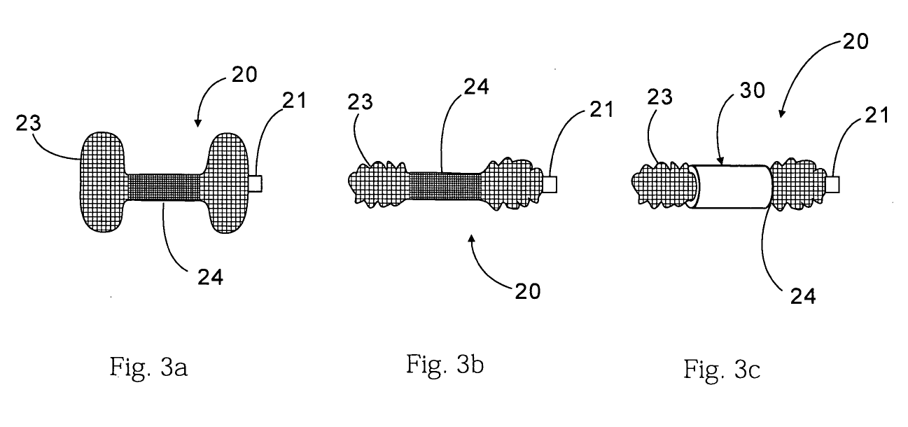

In FIGS. 3a to 3c, the flexible container 20 can further be a pre-formed stretchable flexible container. The two ends 21, 23 of the container 20 have been expanded and shaped before the volume and capacity of the container are shrunk, creating a difference between the expansion coefficient of the two ends and the expansion coefficient of the middle section 24 of the flexible container 20, so that an effect of different volume expansions of the two ends and the middle section of the flexible container 20 is achieved when the medical filling 60 is infused.

Moreover, when the flexible container 20 further is received in the tubular component 30, the middle section 20 will further be restricted by the tubular component 30, and the two ends 21, 23 exposing from the two ends of the tubular component 30 will have a larger difference in the expansion effect.

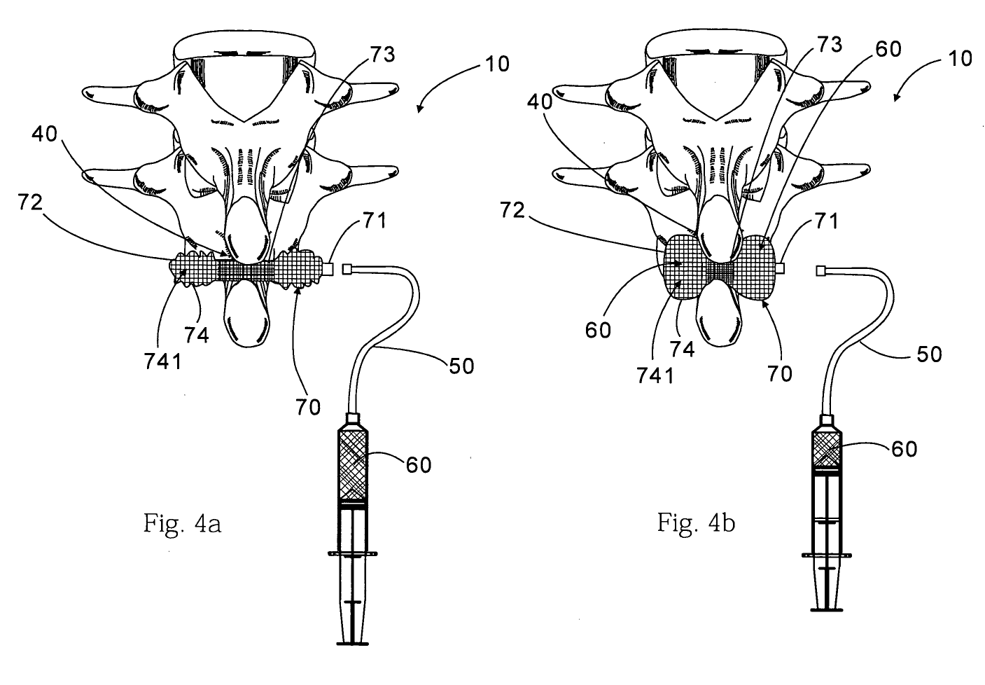

In FIGS. 4a and 4b, a medical implantation device for spine 10 constructed according to another preferred embodiment of the present invention is shown, which includes a pre-formed stretchable flexible container 70, wherein an infusion end 71 and the other end 72 of the flexible container 70 are pre-expanded and shaped, and then shrunk, so that the expansion coefficient of the two ends is different from the expansion coefficient of the middle section 73 of the container 70.

The container 70 has one or more layers of flexible peripheral wall 74. The peripheral wall 74 has plural through holes 741 thereon for communicating the inside of the container 70 with the outside of the container 70, and the container 70 is used to be implanted between two adjacent spinous processes 40.

Moreover, the pore size of the through holes 741 on the peripheral wall of the flexible container 70 is also smaller than the particle size of the medical filling 60 infused by the infusion tool 50. During an infusion operation, the medical filling 60 is infused from the infusion end 71 to inflate the flexible container 70. Meanwhile, the longest distance between two points on the cross-section of the middle section 73 after expansion is larger than the distance between the two adjacent spinous processes 40 implanted with the container 70, and the two ends 71, 72 of the contained are inflated and movably pressed on the two sides of the spinous process 40.

The size of the pores 741 on the peripheral wall of the flexible container 70 is substantially smaller than 100 meshes, and the flexible container 70 and the infusion tool 50 can be connected or disconnected by threading.

The medical filling 60 is, for example, a bone cement (e.g. poly(methyl methacrylate) (PMMA)), or a bone substitute (e.g. gypsum, calcium phosphates), or a silicone filling applicable on bone tissues. The medical filling 60 is of a slurry form prior to the operation or is prepared into a slurry form shortly before the operation, and is infused into the flexible container 70 by the infusion tool 50, and is developed into solid or an elastic semi-solid after the operation.

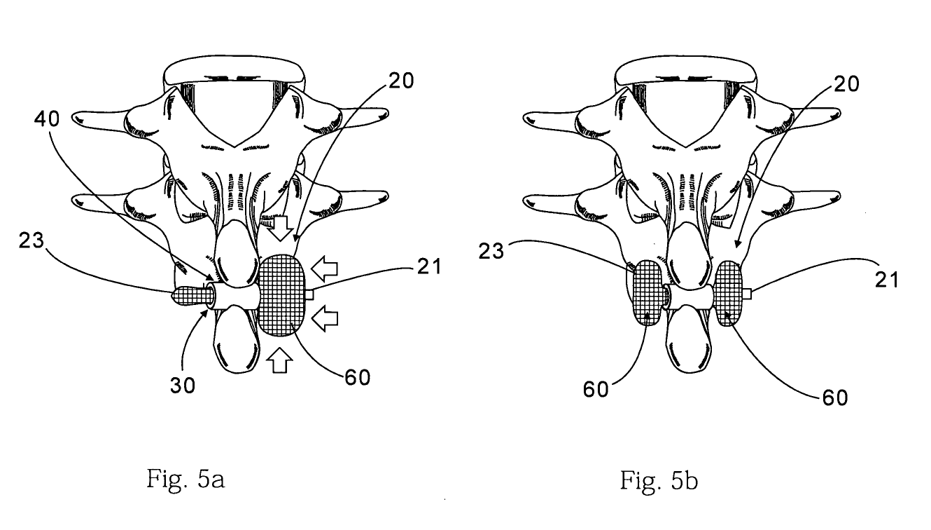

In FIG. 5a and FIG. 5b, after the flexible container 20 and the tubular component 30 of the medical implantation device for spine are assembled, the medical filling 60 is infused into the infusion end 21 of the flexible container 20, and then the infusion end 21 is sealed. Next, the assembly of the container 20 and the tubular component 30 is implanted between two adjacent spinous processes 40. At this time, the infusion end 21 of the container 20 exhibits a greatly expanded condition, and the other end 23 is on the other side of the spinous processes.

A force is applied on the infusion end 21, so that the medical filling 60 is moved gradually to the other end 23. In the meantime, the middle section of the flexible container 20 is restricted by the tubular component 30, and the two ends 21, 23 are inflated and movably pressed on the two sides of the spinous processes 40.

Moreover, the components in FIGS. 5a and 5b are all implemented the same way as in the above-mentioned preferred embodiments.

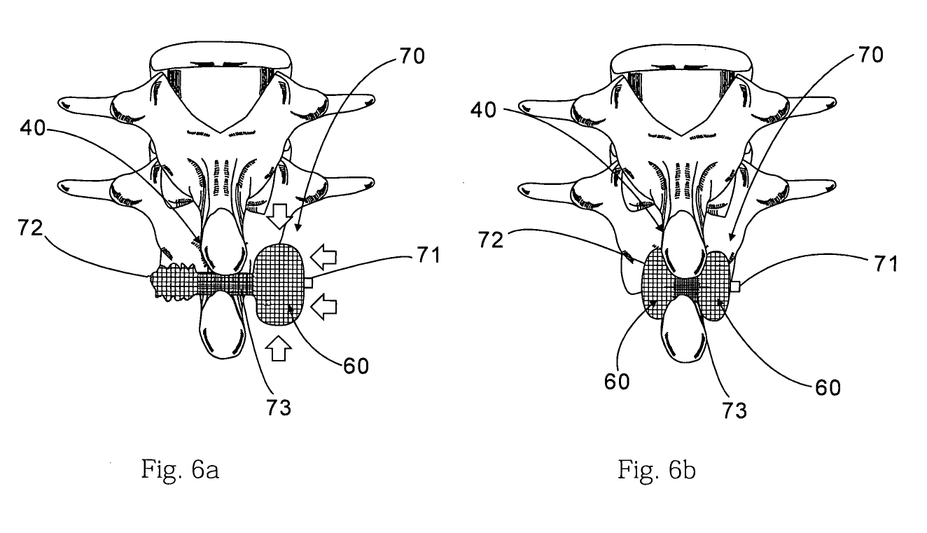

In FIG. 6a and FIG. 6b, the flexible container 70 is the same as the one shown in FIGS. 4a and 4b; however, the infusion end 71 of the flexible container 70 is sealed after infusion of the medical filling 60. Furthermore, the container 70 with the resulting inflated infusion end 71 is implanted between two adjacent spinous processes 40 with the other end 72 thereof being exposed from the other side of the spinous processes 40. After implantation, a force is applied on the infusion end 71 to push and move the medical filling 60 into the other end 72, so that the other end 72 is inflated. Similar to the embodiment shown in FIGS. 4a and 4b, the longest distance between two points on the cross-section of the middle section 73 after inflation is larger than the distance between the two adjacent spinous processes 40 implanted with the container 70 to expand the two spinous processes 40, and the two inflated ends 71, 72 are movably pressed on the two sides of the spinous processes 40.

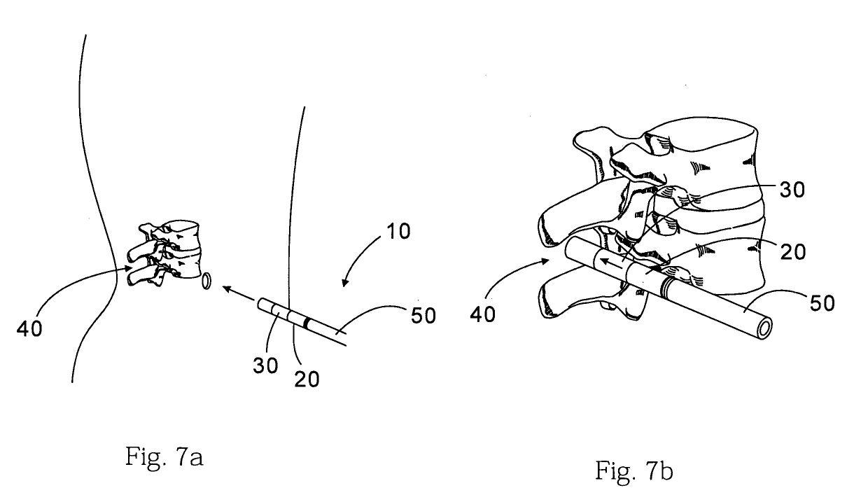

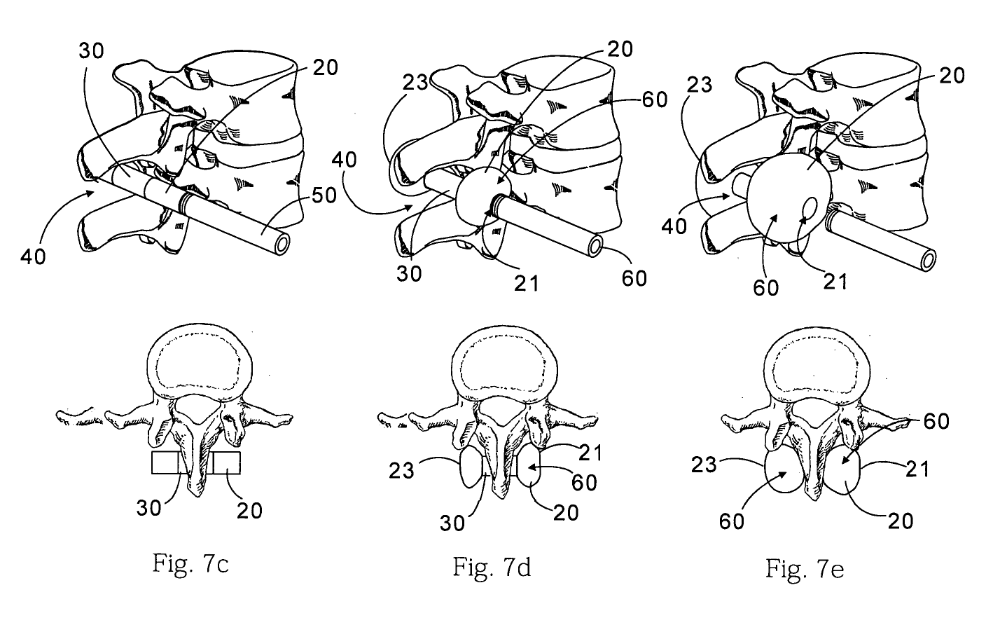

FIG. 7a to FIG. 7e show how the medical implantation device for spine 10 is actually applied on a spinal implantation treatment for a patient. During the operation, a small incision is opened near a diseased spot of the patient's spine. The prepared assembly of the flexible container 20 and the tubular component 30 is carefully aligned with the diseased spot of the spine and is inserted into a space between two adjacent spinous processes 40. X-ray is used to confirm whether the medical implantation device for spine 10 has been implanted to a correct position between the two adjacent spinous processes 40. At this time, the two spinous processes 40 are propped open with the tubular component 30, and the problem of spinal compression has been solved preliminarily.

Subsequently, the medical filling 60 is slowly and stably infused into the flexible container 20 so that the two ends 21, 23 of the flexible container 20 are gradually inflated. The two ends 21, 23 of the flexible container 20 are confirmed whether they are inflated after a predetermined amount of the medical filing has be infused, and the infusion is continued until the inflated ends 21, 23 press on the two sides of the spinous processes 40. The medical filling 60 is allowed to solidify after several minutes of waiting, and then the infusion tool 50 is rotated carefully to be detached from the flexible container 20 and withdrawn from the patient's body. After sterilization, the incision is closed by sewing, thereby completing the operation application of the medical implantation device for spine 10.

1. A medical implantation device for spine comprising
a flexible container with one end thereof being provided with an infusion end, wherein the container has one or more layers of flexible peripheral wall, and the peripheral wall has a plurality of through holes thereon for communicating an inside of the container with an outside of the container; and
a tubular component, wherein the flexible container is inserted into the tubular component with the infusion end and another end of the flexible contained exposing from two ends of the tubular component, and the tubular component is to be implanted between two adjacent spinous processes;
wherein the tubular component has an outer diameter greater than a distance between the two adjacent spinous processes, the through holes on the peripheral wall of the flexible container have a size smaller than a particle size of a medical filling to be infused into the flexible container via the infusion end thereof, wherein an inflation of a middle section of the flexible container is restricted by the tubular component and the infusion end and the another end of the flexible container are inflated to be movably pressed on the two sides of the two adjacent spinous processes, when the medical filling is infused into the flexible container.

2. The medical implantation device for spine as claimed in claim 1, wherein said flexible container is a preformed stretchable flexible container, the infusion end and the another end of the container are expanded and shrunk to have an expansion coefficient different from that of a middle section of the container.

3. The medical implantation device for spine as claimed in claim 1, wherein the size of the through holes on the peripheral wall of the flexible container is smaller than 100 mesh.

4. The medical implantation device for spine as claimed in claim 1 further comprising an infusion tool disconnectably connected to the infusion end of the flexible container, and the infusion tool is capable of storing and infusing the medical filling.

5. The medical implantation device for spine as claimed in claim 4, wherein the infusion end of the flexible container and a front end of the infusion tool are provided with threads for disconnectable connection with each other.

6. The medical implantation device for spine as claimed in claim 1, wherein the medical filling is prepared into a slurry form before operation, infused into the flexible container through an infusion tool, and becomes solid after operation.

7. The medical implantation device for spine as claimed in claim 6, wherein the medical filling is a bone cement for bone tissues or a bone substitute.

8. The medical implantation device for spine as claimed in claim 1, wherein the medical filling is prepared into a slurry form before operation, infused into the flexible container through an infusion tool, and becomes an elastic semi-solid after operation.

9. The medical implantation device for spine as claimed in claim 8, wherein the medical filling is a silicone filling applicable on bone tissues.

10. A medical implantation device for spine comprising a pre-formed stretchable flexible container having an infusion end and another end, wherein the infusion end and the another end of the container are expanded and shrunk to have an expansion coefficient different from that of a middle section of the container, wherein the container comprises one or plural layers of flexible peripheral wall, wherein the peripheral wall is provided with a plurality of through holes for communicating an inside of the container with an outside of the container, and the container is to be implanted between two adjacent spinous processes;
wherein the through holes of the peripheral wall of the flexible container is smaller than a particle size of a medical filling to be infused from the infusion end to inflate the flexible container, wherein a longest distance between two points on a cross-section of the resulting inflated middle section is greater than a distance the two adjacent spinous processes, and the resulting inflated infusion end and the resulting inflated another end of the flexible container are movably pressed on the two sides of the two adjacent spinous processes.

11. The medical implantation device for spine as claimed in claim 10, wherein the size of the through holes on the peripheral wall of the flexible container is smaller than 100 mesh.

12. The medical implantation device for spine as claimed in claim 10 further comprising an infusion tool disconnectably connected to the infusion end of the flexible container, and the infusion tool is capable of storing and infusing the medical filling.

13. The medical implantation device for spine as claimed in claim 12, wherein the infusion end of the flexible container and a front end of the infusion tool are provided with threads for disconnectable connection with each other.

14. The medical implantation device for spine as claimed in claim 10, wherein the medical filling is prepared into a slurry form before operation, infused into the flexible container through an infusion tool, and becomes solid after operation.

15. The medical implantation device for spine as claimed in claim 14, wherein the medical filling is a bone cement for bone tissues or a bone substitute.

16. The medical implantation device for spine as claimed in claim 10, wherein the medical filling is prepared into a slurry form before operation, infused into the flexible container through an infusion tool, and becomes an elastic semi-solid after operation.

17. The medical implantation device for spine as claimed in claim 16, wherein the medical filling is a silicone filling applicable on bone tissues.

18. A medical implantation device for spine comprising:
a flexible container with one end thereof being provided with an infusion end, wherein the container has one or more layers of flexible peripheral wall, and the peripheral wall has a plurality of through holes thereon for communicating an inside of the container with an outside of the container;
a tubular component, wherein the flexible container is inserted into the tubular component with the infusion end and another end of the flexible contained exposing from two ends of the tubular component, and the tubular component is to be implanted between two adjacent spinous processes; and
a medical filling to be infused into the flexible container via the infusion end thereof,
wherein the tubular component has an outer diameter greater than a distance between the two adjacent spinous processes, the through holes on the peripheral wall of the flexible container have a size smaller than a particle size of the medical filling, wherein the infusion end of the flexible container is sealed following completion of the infusion of the medical filling, the tubular component with the sealed flexible container are implanted between two adjacent spinous processes, and a force is applied on the infusion end to push and move the medical filling from the infusion end to the another end of the flexible container while an inflation of a middle section of the flexible container is restricted by the tubular component, so that the infusion end and the another end of the flexible container are inflated to be movably pressed on the two sides of the two adjacent spinous processes.

19. The medical implantation device for spine as claimed in claim 18, wherein said flexible container is a pre-formed stretchable flexible container, the infusion end and the another end of the container are expanded and shrunk to have an expansion coefficient different from that of a middle section of the container.

20. The medical implantation device for spine as claimed in claim 18, wherein the size of the through holes on the peripheral wall of the flexible container is smaller than 100 mesh.

21. The medical implantation device for spine as claimed in claim 18 further comprising an infusion tool disconnectably connected to the infusion end of the flexible container, and the infusion tool is capable of storing and infusing the medical filling.

22. The medical implantation device for spine as claimed in claim 21, wherein the infusion end of the flexible container and a front end of the infusion tool are provided with threads for disconnectable connection with each other.

23. The medical implantation device for spine as claimed in claim 18, wherein the medical filling is prepared into a slurry form before operation, infused into the flexible container through an infusion tool, and becomes solid after operation.

24. The medical implantation device for spine as claimed in claim 23, wherein the medical filling is a bone cement for bone tissues or a bone substitute.

25. The medical implantation device for spine as claimed in claim 18, wherein the medical filling is prepared into a slurry form before operation, infused into the flexible container through an infusion tool, and becomes an elastic semi-solid after operation.

26. The medical implantation device for spine as claimed in claim 25, wherein the medical filling is a silicone filling applicable on bone tissues.

27. A medical implantation device for spine comprising:
a pre-formed stretchable flexible container having an infusion end and another end, wherein the infusion end and the another end of the container are expanded and shrunk to have an expansion coefficient different from that of a middle section of the container, wherein the container comprises one or plural layers of flexible peripheral wall, wherein the peripheral wall is provided with a plurality of through holes for communicating an inside of the container with an outside of the container, and the container is to be implanted between two adjacent spinous processes; and
a medical filling to be infused into the flexible container via the infusion end thereof,
wherein the through holes on the peripheral wall of the flexible container have a size smaller than a particle size of the medical filling, wherein the infusion end of the flexible container is sealed following completion of the infusion of the medical filling, the sealed flexible container is implanted between two adjacent spinous processes, and a force is applied on the infusion end to push and move the medical filling from the infusion end to the another end of the flexible container, so that an inflation of a middle section of the flexible container has a longest distance between two points on a cross-section of the inflated middle section greater than a distance between the two adjacent spinous processes, and the infusion end and the another end of the flexible container are inflated to be movably pressed on the two sides of the two adjacent spinous processes.

28. The medical implantation device for spine as claimed in claim 27, wherein the size of the through holes on the peripheral wall of the flexible container is smaller than 100 mesh.

29. The medical implantation device for spine as claimed in claim 27 further comprising an infusion tool disconnectably connected to the infusion end of the flexible container, and the infusion tool is capable of storing and infusing the medical filling.

30. The medical implantation device for spine as claimed in claim 29, wherein the infusion end of the flexible container and a front end of the infusion tool are provided with threads for disconnectable connection with each other.

31. The medical implantation device for spine as claimed in claim 27, wherein the medical filling is prepared into a slurry form before operation, infused into the flexible container through an infusion tool, and becomes solid after operation.

32. The medical implantation device for spine as claimed in claim 31, wherein the medical filling is a bone cement for bone tissues or a bone substitute.

33. The medical implantation device for spine as claimed in claim 27, wherein the medical filling is prepared into a slurry form before operation, infused into the flexible container through an infusion tool, and becomes an elastic semi-solid after operation.

34. The medical implantation device for spine as claimed in claim 33, wherein the medical filling is a silicone filling applicable on bone tissues.
